(19) United States
(12) Patent Application Publication
(10) Pub. No.: US 2020/0315454 A1
(43) Pub. Date: Oct. 8, 2020

(54) LOCATION BASED ECG ANALYSIS CRITERIA TUNING IN ECG EQUIPMENT

(71) Applicant: KONINKLIJKE PHILIPS N.V., EINDHOVEN (NL)

(72) Inventor: RICHARD EARL GREGG, WESTFORD, MA (US)

(21) Appl. No.: 16/955,117

(22) PCT Filed: Dec. 18, 2018
(86) PCT No.: PCT/EP2018/085366

§ 371 Date: Jun. 18, 2020

Related U.S. Application Data
(60) Provisional application No. 62/607,949, filed on Dec. 20, 2017.

(51) Int. Cl. A61B 5/00; A61B 5/04; A61B 5/0432; A61B 5/046; G16H 40/67; G16H 40/20

(52) U.S. Cl. CPC A61B 5/002; A61B 5/04012; A61B 5/0432; G16H 40/20; A61B 5/0006; A61B 5/746; G16H 40/67; A61B 5/046

(57) ABSTRACT

A cardiac device includes an electrocardiograph (ECG device) (10, 110), an electronic processor (12, 36), and a non-transitory storage medium (14, 38) storing instructions readable and executable by the electronic processor to perform a process including: receiving a location of the ECG device; tuning one or more ECG diagnostic criteria for the received location to generate one or more ECG analysis criteria tuned for the received location; operating the ECG device to acquire one or more ECG traces; and performing cardiac analysis by applying the one or more ECG analysis criteria tuned for the received location to the one or more ECG traces to generate cardiac analysis information. The tuning may include associating the ECG device with a medical department based on the location and tuning the one or more ECG analysis criteria for the associated medical department. Alternatively, the associated medical department may be received directly.

### FIELD

The following relates generally to the electrocardiograph (ECG) arts, patient monitoring arts, ECG monitoring arts, patient data management arts, and related arts.

### BACKGROUND

Mobile electrocardiograph (ECG) devices, such as cart-based 12 lead ECGs, plug-in 2 or 3 lead ECG units for connection with a patient monitor, stress ECG systems, and telemetry monitors, advantageously can be flexibly deployed depending upon current clinical need. Flexible deployment is facilitated by ECG devices typically having similar operation regardless of the clinical setting in which they are deployed. The ECG device collects ECG data, applies various ECG analysis criteria to the collected ECG data to detect clinical abnormalities, and outputs the ECG traces as trend lines, displays clinical abnormalities detected by the ECG analysis criteria and may also issue audible and visible alarms to indicate clinical abnormalities of importance.

A diagnostic ECG device records a snapshot (usually a 10 second time interval) of the patient's cardiac activity. The standard diagnostic ECG device has a 12-lead configuration with 10 electrodes giving 12 different “electrical” views (called “leads”) of the heart. Once recorded, the diagnostic ECG device analyzes the recorded ECG traces for the 12 leads and applies various ECG diagnostic criteria to arrive at an interpretation of the diagnostic ECG device. Commonly, a cardiologist or other medical professional with suitable expert knowledge reviews the diagnostic ECG device and prepares a written report that is stored in the patient's Electronic Medical Record (EMR) or the like together with the traces for each of the 12 leads, and may be printed out or otherwise used. The twelve views or leads of the standard diagnostic ECG device advantageously provide substantial cardiac diagnostic information, and can be utilized by a skilled cardiologist to diagnose a wide range of specific cardiac deficiencies or ailments.

Another type of ECG device is a monitoring ECG device, which streams ECG traces in real-time. A monitoring ECG device often employs fewer electrodes and consequently provides fewer views or leads, in some embodiments employing as few as two electrodes providing a single ECG trace. A monitoring ECG device may be a standalone device, or may be a plug-in module connecting with (or an integral component of) a multifunction patient monitor. The monitoring ECG signals are analyzed, displayed and recorded in real-time. The purpose of a monitoring ECG device is typically not to diagnose specific cardiac deficiencies or ailments, but rather to detect serious life threatening conditions such as myocardial infarction (colloquially, a heart attack), cardiac arrest (cessation of cardiac function), excessively fast or slow heart rate, or so forth, and raise an alarm quickly. Monitoring ECG devices analyze the ECG signal to measure cardiac parameters, apply alarm thresholds to those parameters and alarm if the parameter is outside the desired range indicating a condition that needs clinical attention.

ECG analysis criteria depend upon the purpose of the ECG device, that is, the task being performed by the ECG device. In the case of a diagnostic ECG device, the ECG analysis criteria typically identify particular cardiac deficiencies or ailments based on the substantial information provided by the 12 leads of a standard 12-lead ECG device. Two non-limiting illustrative examples of ECG analysis criteria include STEMI criteria and atrial fibrillation criteria. STEMI criteria detect ST elevations that are potentially indicative of acute myocardial infarction. An example of an atrial fibrillation diagnostic criteria is irregular RR intervals and an absence of P-waves. ECG analysis criteria in the context of diagnostic ECG device (also sometimes referred to herein as ECG diagnostic criteria) can be complex: for example, STEMI criteria may be satisfied by ST elevations in certain leads, or combinations of leads, exceeding specified threshold values (e.g. in mV or mm using conventional units), and may further provide diagnostic analysis for distinguishing the type of abnormality (e.g. a specific bundle branch block). It may be noted that the term “ECG diagnostic criterion” or similar phraseology does not imply automated diagnosis—rather, diagnostic information output by a diagnostic ECG device is preferably reviewed by a cardiologist or other appropriate medical professional who then promulgates a diagnosis.

In the case of monitoring ECG device, the ECG analysis criteria are typically expressed as thresholds, e.g. generating an alarm if the heart rate exceeds or falls below specified thresholds. These thresholds may depend on patient-specific factors such as age or sex.

The following discloses certain improvements.

### SUMMARY

In some embodiments disclosed herein, a cardiac device comprises an electrocardiograph (ECG device), an electronic processor, and a non-transitory storage medium that stores instructions readable and executable by the electronic processor to perform a process including: receiving a location of the ECG device; tuning one or more ECG analysis criteria for the received location to generate one or more ECG analysis criteria tuned for the received location; operating the ECG device to acquire one or more ECG traces; and performing cardiac analysis by applying the one or more ECG analysis criteria tuned for the received location to the one or more ECG traces to generate cardiac analysis information.

In some embodiments disclosed herein, a cardiac method is disclosed. A location of an ECG device is received. The ECG device is associated with a medical department based on the received location. One or more ECG analysis criteria of the ECG device are tuned for the medical department with which the ECG device is associated to generate one or more ECG analysis criteria tuned for the associated medical department. The ECG device is operated to acquire one or more ECG traces, and cardiac analysis is performed by applying the one or more ECG analysis criteria tuned for the associated medical department to the one or more ECG traces to generate cardiac analysis information. The receiving, associating, tuning, operating, and performing of cardiac analysis are suitably performed by one or more electronic processors.

In some embodiments disclosed herein, a non-transitory storage medium stores instructions readable and executable by an electronic processor to perform a method including associating an ECG device with a medical department. One or more ECG analysis criteria of the ECG device are tuned for the medical department with which the ECG device is associated to generate one or more ECG analysis criteria tuned for the associated medical department. Cardiac analysis is performed by applying the one or more ECG analysis criteria tuned for the associated medical department to one or more ECG traces acquired by the ECG device to generate cardiac analysis information.

One advantage resides in facilitating flexible deployment of ECG devices across departments or other medical units of a hospital or other medical facility that employ different ECG analysis criteria and/or assign different significances to certain clinical abnormalities detected by ECG analysis criteria.

Another advantage resides in providing for efficient updating of the ECG analysis criteria of ECG devices deployed across a hospital or other medical facility.

Another advantage resides in providing for implementation of different ECG analysis criteria when ECG devices are deployed in different medical departments or medical units of a hospital or other medical facility.

Another advantage resides in reduced false alarms being issued by ECG devices.

Another advantage resides in more efficient tuning of ECG analysis criteria of an ECG device.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a monitoring ECG device deployed in a medical facility and further illustrating associated structures.

FIG. 2 diagrammatically illustrates a diagnostic ECG device deployed in a medical facility and further illustrating associated structures.

FIG. 3 diagrammatically illustrates a method of tuning the ECG analysis criteria of the monitoring ECG device of FIG. 1.

FIG. 4 diagrammatically illustrates a method of tuning the ECG analysis criteria of the diagnostic ECG device of FIG. 2.

### DETAILED DESCRIPTION

Flexible deployment of ECG devices across medical departments or other medical units of a hospital or other medical facility advantageously enables such equipment to be efficiently utilized in a cost-effective manner. However, it is recognized herein that deployment of ECG devices across medical departments can be problematic due to differences in the demographics of the patient populations served by various medical departments. The significance of various ECG analysis criteria may differ depending on the deployment environment. For example, atrial fibrillation detected in a general ward patient may be of lesser concern than atrial fibrillation detected in a post-operative patient, and may be of most concern in a post-operative cardiac surgery patient. To be safe, ECG manufacturers usually tune the ECG analysis criteria aggressively, that is, for the situation in which the clinical abnormality is most serious (in the instant example, for the post-operative cardiac surgery patient). This can lead to overly sensitive detection of clinical abnormalities in other deployment environments, and an undesirably high rate of false alarms. On the other hand, if the ECG diagnostic criteria are tuned less aggressively then the ECG device may fail to provide timely alerts of abnormal cardiac conditions.

One approach to addressing this concern might be to tune the ECG analysis criteria for a particular patient as prescribed by a physician or per guidelines promulgated by the particular medical department to which the patient is assigned. However, this requires that the proper ECG analysis criteria tuning be selected at the time the ECG device is set up for the patient. In the case of monitoring ECG device, the information must also be updated each time the patient is transferred to a new department. This can occur frequently for some patients: for example, a patient may be initially admitted to the emergency room, then transferred to a cardiac care unit (CCU), then to a pre-operative ward, then to a cardiac surgery department, then to a post-operative ward, then to a further step-down ward or a general ward. Each such transfer may warrant updating the ECG analysis criteria, which would be time consuming for clinicians.

In disclosed embodiments, the ECG analysis criteria are tuned based on the location of the ECG device in the hospital, or by the medical department to which the ECG device is assigned (which may optionally be determined from the location of the ECG device). This advantageously permits automatic tuning, including automatic updating as the patient's situation changes (as reflected by changes in patient's location, e.g. from a pre-operative ward to a post-operative ward, or by transfer of the patient between medical departments). It also allows for efficient updating of ECG analysis criteria as best clinical practices evolve for various medical departments. The location of the ECG device can be variously determined. In one approach, a Real Time Locating Service (RTLS) is used to directly track the location of the ECG device in real time. However, this may be susceptible to difficulties if the patient is ambulatory or transported by medical personnel. In another approach, it is recognized that the appropriate ECG analysis criteria tuning is often best chosen by the currently assigned location of the ECG device, e.g. the medical department or nurses' station to which the ECG device is assigned.

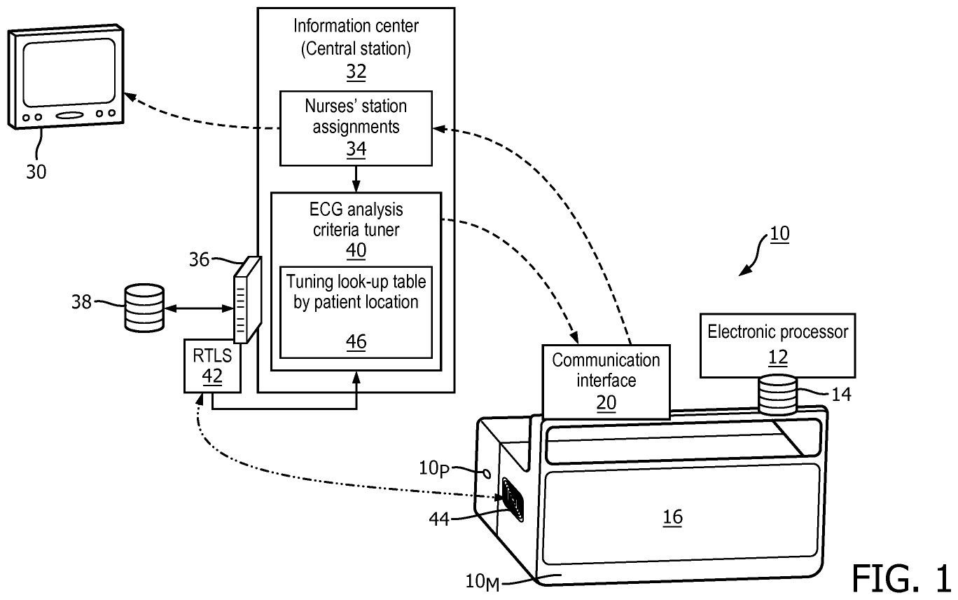

With reference to FIG. 1, a monitoring cardiac monitoring device includes a monitoring electrocardiograph (ECG device) 10. The illustrative monitoring ECG device 10 is implemented as a plug-in ECG module 10_{P} that plugs into a mating receptacle of a mobile monitor 10_{M} that may provide other monitoring capabilities (e.g. SpO₂, respiratory monitoring, et cetera). In other embodiments, the monitoring ECG device may be embodied as a standalone ECG device unit, or as an integral component of a multifunction mobile monitor. The monitoring ECG device 10 includes an electronic processor 12 and a non-transitory storage medium 14 storing instructions readable and executable by the electronic processor 12 to perform a cardiac monitoring process. (Note, these components 12, 14 are typically internal to the monitoring ECG device 10 and are indicated diagrammatically in FIG. 1). The illustrative monitoring ECG device 10 also includes a display 16 for displaying ECG traces (and other acquired vital sign data in the case of a multifunction mobile monitor 10_{M}), and at least one user input device implemented, for example, as a touch-sensitive overlay of the display 16 or via a Bluetooth™ link with a cellphone or as a built-in keypad (not shown) or so forth. The monitoring ECG device 10 is typically a 2 or 3 lead ECG device, though provision for more than 3 leads is contemplated, e.g. a 12-lead monitoring ECG device is contemplated. A cardiac monitoring process performed by the electronic processor 12 includes operating the monitoring ECG device 10 to acquire one or more ECG traces. To this end, the monitoring ECG device 10 is connected with a monitored subject (e.g. hospital patient or the like) using two or more ECG electrodes (not shown) connected to define one or more ECG leads whose ECG traces are measured.

The monitoring ECG device 10 optionally further includes a communication interface 20 for transmitting ECG traces, and optionally also cardiac diagnostic information derived from the ECG traces, to a monitoring location such as an illustrative nurses' station 30. In the illustrative setup, an information center or central station 32 serves as an intermediary for this communication. By way of non-limiting illustrative example, the information center or central station 32 may be embodied as a Philips IntelliVue™ Information Center Central monitoring system (available from Koninklijke Philips N.V.). The information center or central station 32 collects real-time patient data and distributes it to nurses' stations or other monitoring locations, and/or records the patient data in an Electronic Medical Record (EMR), Electronic Health Record (EHR), Cardiovascular Information System (CVIS), or other electronic patient database(s), and/or communicates the patient data to a Clinical Decision Support (CDS) system, or otherwise stores and/or presents and/or utilizes the patient data. The communication interface 20 of the monitoring ECG device 10 via which ECG traces and/or cardiac diagnostic information is offloaded from the monitoring ECG device 10 (and, in some embodiments, via which information is communicated to the monitoring ECG device 10) may be any suitable wired or wireless electronic communication interface: by way of some non-limiting illustrative examples, the communication interface 20 may be a wired Ethernet connection, a wireless WiFi connection, or so forth; or may be a plug-in connector coupling to a host patient monitor in the case of a plug-in ECG unit; or may be a USB port, Bluetooth™, or other connection in the case of a telemetry monitor; or so forth. A given ECG device may include two or more communication interfaces to promote flexible wired and/or wireless connectivity. At the illustrative information center or central station 32, a nurses' station assignments table or data structure 34 identifies the nurses' station to which the monitoring ECG device 10 is assigned. This may be a direct association, e.g. a table identifying the monitoring ECG device 10 by network ID or the like associated with a nurses' station, or may be an indirect association by way of the patient, i.e. the monitoring ECG device 10 may be assigned to a patient who in turn is assigned to a nurses' station. As diagrammatically indicated in FIG. 1, the information center or central station 32 is implemented on a suitable electronic data processing device such as an illustrative server computer 36 which reads and executes instructions implementing the information center or central station 32 which are stored on a non-transitory storage medium 38.

The various non-transitory storage media 14, 38 disclosed herein may comprise various types of computer-readable non-transitory storage media known in the art, such as by way of non-limiting illustrative example: a hard disk drive, RAID array or other magnetic storage medium; a solid state drive (SSD), flash memory or other electronic storage medium; an optical disk or other optical storage medium; various combinations thereof; and/or so forth. The various electronic processors 12, 36 disclosed herein may be variously embodied, for example the electronic processor 12 of the ECG device 10 may be a microprocessor and associated electronics (e.g. RAM, ROM, wired circuitry, or so forth), while the server computer 36 may be a single server computer, a plurality of networked server computers, an ad hoc collection of network-based computers defining a cloud computing resource, and/or so forth.

With continuing reference to FIG. 1, an ECG analysis criteria tuner 40 is implemented at the information center or central station 32, as illustrated, or alternatively is implemented at the monitoring ECG device 10 by the electronic processor 12. The ECG analysis criteria tuner 40 tunes one or more ECG analysis criteria for a location of the monitoring ECG device 10 to generate one or more ECG analysis criteria tuned for the location of the monitoring ECG device 10. The location of the monitoring ECG device 10 is received at the processor 36 (or at the processor 12 if the tuner 40 is implemented at the monitoring ECG device 10) from a suitable source.

In one embodiment, the location of the monitoring ECG device 10 is received from a real time locating service (RTLS) 42. By way of non-limiting illustration, some examples of RTLS technologies include RFID-based RTLS employing RFID tags disposed on or in tracked equipment (for example, an RFID tag 44 disposed on or in the monitoring ECG device 10); WiFi based positioning (WPS) leveraging signal strength of WiFi access point (AP) connections with WiFi-enabled mobile devices; various combinations thereof; or so forth. In the illustrative example, the RTLS 42 is RFID-based, and RFID tag readers (not shown) disposed at strategic placements around the hospital or other medical facility detect the RFID tag 44. An inventory database of the RTLS 42 associates the RFID 44 with the monitoring ECG device 10, and an electronic map of the hospital or other medical facility identifies the location based on which RFID tag reader picks up the RFID tag 44 or, in a more advanced embodiment, detection of the RFID tag 44 by two or three RFID tag readers enables more precise location by way of triangulation.

In another embodiment, the location is received as an identification of the assigned nurses' station 30 to which the monitoring ECG device 10 is assigned. For example, this assignment may be stored in the nurses' station assignments database 34. In another contemplated embodiment (not shown), the location is received via a user input device of the monitoring ECG device 10.

The location of the monitoring ECG device 10 received from the RTLS 42 or from the nurses' station assignments database 34 may optionally be used to associate the monitoring ECG device 10 with a medical department based on the received location. For example, if the location of the monitoring ECG device 10 received from the RTLS 42 is within the boundaries of the cardiology department then this may be used to associate the monitoring ECG device 10 with the cardiology department; whereas, if the ECG device location received from the RTLS 42 is within the boundaries of a post-operative ward or department then this may be used to associate the monitoring ECG device 10 with the post-operative department; whereas, if the ECG device location received from the RTLS 42 is within the boundaries of a surgical ward then this may be used to associate the monitoring ECG device 10 with the surgery department; and so forth. In embodiments in which the location is received from the nurses' station assignments database 34, the monitoring ECG device 10 is suitably associated with the medical department of which the assigned nurses' station is a part.

In a variant embodiment, the medical department is directly received rather than being determined from a received location. For example, the medical department may be received from an electronic hospital inventory stored on the non-transitory storage medium 38, e.g. maintained by a hospital inventorying system. In another example, the user inputs the medical department directly using a user input device of the monitoring ECG device 10.

To perform the tuning, the ECG analysis criteria tuner 40 inputs the location or information derived from the location (e.g. the medical department to which the ECG device 10 is associated) to a tuning look-up table 46 that associates location-specific ECG analysis criteria with specific locations (or medical departments associated with the locations). The tuning information is sent to the monitoring ECG device 10 via the communication interface 20, where the appropriate ECG analysis criteria are set up. In alternative embodiments in which the tuner 40 is implemented at the monitoring ECG device 10 (not illustrated), the output of the tuner 40 may be directly applied by the monitoring ECG device 10.

In operation, the electronic processor 12 operates the monitoring ECG device 10 to acquire one or more ECG traces, and performs cardiac analysis by applying the one or more ECG analysis criteria tuned for the received location (or medical department derived from the location) to the one or more ECG traces to generate cardiac analysis information.

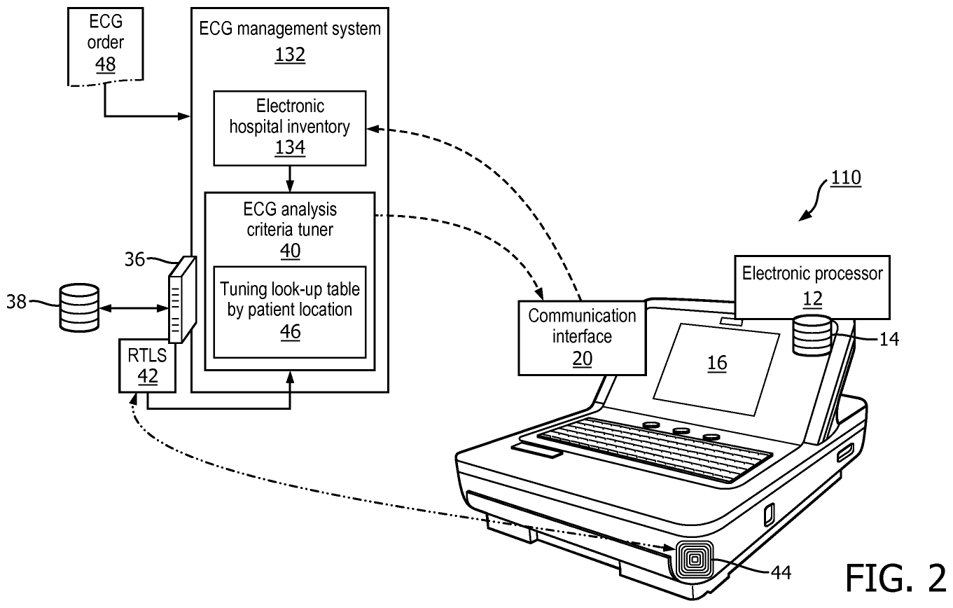

With reference to FIG. 2, a diagnostic ECG device 110 is illustrated. By way of non-limiting illustrative example, the illustrative diagnostic ECG device 10 is a Philips PageWriter TC30™ cardiograph (available from Koninklijke Philips N.V., Eindhoven, the Netherlands), but more generally the disclosed improvements can be incorporated into any diagnostic ECG device. The illustrative diagnostic ECG device 110 includes the components 12, 14, 16, 20 analogous to the monitoring ECG device 10 of FIG. 1, and is tagged by an RFID tag 44 for locating by the RTLS 42. In a conventional twelve-lead configuration, the diagnostic ECG device 110 is electrically connected with ten electrodes positioned in the standard 12-lead pattern on the patient to permit measurement of the standard twelve ECG leads with each ECG lead defined as the voltage difference between a defined two electrodes. As is known in the art, a twelve-lead ECG is a standard lead configuration for detailed diagnostic ECG device; however, other lead configurations are sometimes used for diagnostic ECG device, such as a 16 or 18-lead configuration for high sensitivity STEMI detection.

In the diagnostic ECG setting, the information center or central station 32 is typically replaced by a ECG management system 132 implemented by the computing hardware (e.g. server 36 and non-transitory storage medium 38). The location of the diagnostic ECG device 110 returned by the RTLS 42 detecting the RFID tag 44 (or otherwise determined) is associated with a department by way of an electronic hospital inventory 134, and the ECG analysis tuner 40 again references a suitable lookup table 46 to determine the ECG analysis criteria, which in this case are typically EGC diagnostic criteria. For example, the ECG analysis criteria may include an atrial fibrillation diagnostic criteria, which may be assigned a higher set of thresholds (i.e. less likely for atrial fibrillation to be indicated) if the diagnostic ECG device 110 is associated with a general ward, and may be assigned a lower set of thresholds (i.e. more likely for atrial fibrillation to be indicated) if the diagnostic ECG device 110 is associated with a post-operative cardiac surgery recovery ward where new atrial fibrillation is more prevalent. In this way, the diagnostic ECG device 110 applies an atrial fibrillation diagnostic criteria with a sensitivity to atrial fibrillation that is appropriate for the medical setting of the patient. Another example would be ST-segment depression criteria for the emergency department versus the general ward. The same ST depression that would be considered non-specific in the general ward could be considered abnormal indicating likely cardiac ischemia in the emergency department to make sure acute myocardial infarction is not missed in the Emergency Department. A further example would be QT interval thresholds used for a psychiatric ward because many drugs used in a psychiatric ward pose a threat of prolonging the QT interval thus increasing the risk of sudden cardiac death. Dangerous QT prolongation would be more prevalent in the psychiatric ward, therefore the QT interval thresholds would be reduced for higher sensitivity. The tuning may adjust other parameters of the ECG diagnostic criteria besides thresholds, such adjusting or selecting as the formula or algorithm applied to perform a particular ECG diagnostic, or the form of output (e.g. displayed diagnostic information and audio alarm, or only displayed diagnostic information but no audio alarm), and/or so forth. Furthermore, since the diagnostic ECG device 110 may be capable of performing a range of ECG diagnostics leveraging the extensive capabilities provided by the standard twelve-lead configuration, the diagnostic ECG device may be performed in response to an ECG order 48 issued by a physician, and the ECG management system 132 may operate in accord with the ECG order 48, possibly including the ECG analysis tuner 40 also referencing the ECG order 48 in determining the ECG analysis criteria.

Thus, in some illustrative embodiments a cardiac device includes the ECG device 10, 110, an electronic processor 12, 36, and a non-transitory storage medium 14, 38 storing instructions readable and executable by the electronic processor to perform a process including: receiving a location of the ECG device 10, 110 (e.g. from the RTLS 42 or from the nurses' station assignments database 34 or from the electronic hospital inventory 134); tuning one or more ECG analysis criteria for the received location to generate one or more ECG analysis criteria tuned for the received location; operating the ECG device 10, 110 to acquire one or more ECG traces; and performing cardiac analysis by applying the one or more ECG analysis criteria tuned for the received location to the one or more ECG traces to generate cardiac analysis information. The tuning may include associating the ECG device 10, 110 with a medical department based on the received location, and tuning the one or more ECG analysis criteria for the medical department with which the ECG device 10, 110 is associated to generate one or more ECG diagnostic criteria tuned for the received location. The one or more ECG criteria may include, by way of non-limiting illustrative example, at least STEMI criteria and atrial fibrillation diagnostic criteria. The tuning may include setting at least one abnormal cardiac condition detection threshold of the one or more ECG diagnostic criteria for the received location. The process may further include outputting at least one of a cardiac analysis display and a cardiac analysis audio alarm in accord with the generated cardiac analysis information.

The use of the disclosed location-based tuning does not preclude additionally tuning one or more ECG diagnostic criteria using other tuning criteria. For example, the one or more ECG diagnostic criteria may also be tuned for age, gender, height, weight, body mass index, chronic conditions such as high blood pressure, diabetes, renal failure or any combination of those factors of the monitored patient, so that the one or more ECG diagnostic criteria tuned for the received location are also tuned for the age and/or gender of the monitored patient.

In a variant embodiment, the ECG method may include: associating the ECG device 10, 110 with a medical department; tuning one or more ECG analysis criteria of the ECG device 10, 110 for the medical department with which the ECG device is associated to generate one or more ECG analysis criteria tuned for the associated medical department; and performing cardiac analysis by applying the one or more ECG analysis criteria tuned for the associated medical department to one or more ECG traces acquired by the ECG device 10, 110 to generate cardiac analysis information. The association of the ECG device 10, 110 with a medical department may be done based on location information for the ECG device 10, 110, e.g. received from the RTLS 42 or from the nurses' station assignments database 34. Alternatively, the association of the ECG device 10, 110 with a medical department may be done directly, e.g. based on a medical devices inventory 134 that stores departmental assignments for medical equipment including the ECG device, or by having the user directly enter the medical department via a user interface of the ECG device.

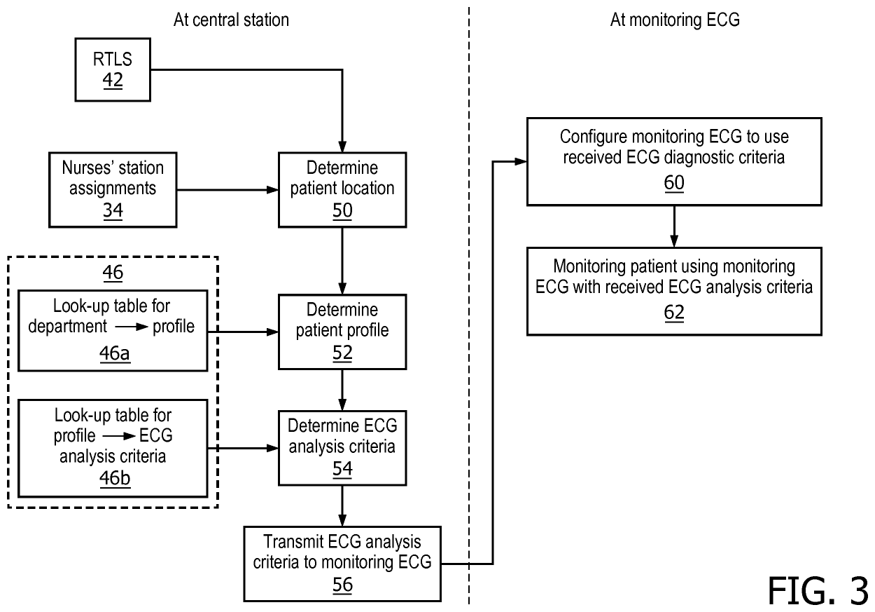

With reference to FIG. 3, an illustrative cardiac monitoring method suitably performed by the cardiac monitoring device of FIG. 1 is illustrated. As shown on the left side of FIG. 3, at the central station 32 the location is determined in an operation 50, e.g. received from the RTLS 42 and optionally converted to a medical department, or received from the nurses' station assignments 34 and again optionally converted to a medical department. The location is input to the tuning look-up table 46 in operations 52, 54 to determine the appropriate location-specific tuning. In the illustrative example of FIG. 3, this is a two-step process: in an operation 52 a department→profile lookup table 46a is referenced to determine a patient profile; and in an operation 54 a department profile→ECG analysis criteria lookup table 46b is referenced to retrieve the ECG analysis criteria. This approach advantageously allows for multiple departments to have the same department profile, which can simplify maintenance. In an operation 56 the ECG analysis criteria tuning information is transmitted to the monitoring ECG device 10 via the communication interface 20. (This step is omitted in the alternative embodiment in which the operations 50, 52, 54 are performed locally at the monitoring ECG device 10 using the electronic processor 12).

With continuing reference to FIG. 3, as shown on the right side, at the monitoring ECG device 10 in an operation 60 the ECG analysis criteria tuning information is received at the monitoring ECG device 10. (Again, this step is omitted in the alternative embodiment in which the operations 50, 52, 54 are performed locally at the monitoring ECG device 10 using the electronic processor 12). Further in the operation 60, the ECG analysis criteria are configured in accord with the received ECG analysis criteria tuning information, for example by setting at least one abnormal cardiac condition detection threshold and/or at least one alarm threshold as indicated in the tuning information. In an operation 62, the monitoring ECG device 10 is operated to generate one or more ECG traces continuously and in real time and to apply the one or more ECG analysis criteria tuned for the received location to the one or more ECG traces to generate cardiac analysis information. This may entail outputting patient alarms if the cardiac analysis information indicates a serious condition requiring immediate intervention by medical personnel, such as a cardiac arrest or a myocardial infarction or an unacceptably fast (or slow) heart rate.

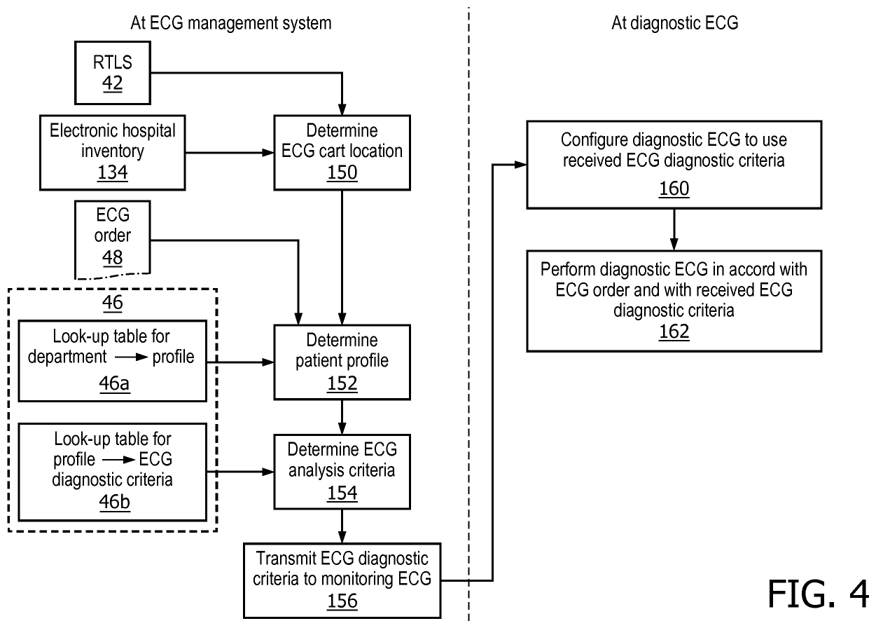

With reference to FIG. 4, an illustrative cardiac diagnostic method suitably performed by the diagnostic cardiac device of FIG. 2 is illustrated. As shown on the left side of FIG. 3, at the ECG management system 132 the location is determined in an operation 150, e.g. received from the RTLS 42 and optionally converted to a medical department, or received from the electronic hospital inventory 134 and again optionally converted to a medical department. The location is input to the tuning look-up table 46 in operations 152, 154 to determine the appropriate location-specific tuning. In the illustrative example of FIG. 4, this is a two-step process: in an operation 152 the department→profile lookup table 46a is referenced to determine a patient profile; and in an operation 154 a department profile→ECG diagnostic criteria lookup table 146b is referenced to retrieve the ECG diagnostic criteria. This approach advantageously allows for multiple departments to have the same department profile, which can simplify maintenance. In an operation 156 the ECG diagnostic criteria tuning information is transmitted to the diagnostic ECG device 110 via the communication interface 20. (This step is omitted in the alternative embodiment in which the operations 150, 152, 154 are performed locally at the diagnostic ECG device 110 using the electronic processor 12).

With continuing reference to FIG. 4, as shown on the right side, at the diagnostic ECG device 110 in an operation 160 the ECG diagnostic criteria tuning information is received at the diagnostic ECG device 110. (Again, this step is omitted in the alternative embodiment in which the operations 150, 152, 154 are performed locally at the monitoring ECG device 110 using the electronic processor 12). Further in the operation 160, the ECG diagnostic criteria are configured in accord with the received ECG diagnostic criteria tuning information, for example by setting at least one abnormal cardiac condition detection threshold and/or at least one alarm threshold as indicated in the tuning information. In an operation 162, the diagnostic ECG device 110 is operated to generate one or more (typically twelve) ECG traces continuously and in real time and to apply the one or more ECG diagnostic criteria tuned for the received location to the one or more ECG traces to generate cardiac diagnostic information. This information along with the ECG traces are typically later reviewed by a cardiologist who prepares a diagnostic ECG report on the patient.

In the diagnostic ECG device and method embodiments shown in FIGS. 2 and 4, the ECG diagnostic criteria are applied at the diagnostic ECG device 110, and the cardiac diagnostic information are sent back to the ECG management system 132. To implement this approach, the operation 156 (FIG. 4) sends the ECG diagnostic criteria tuning information to the diagnostic ECG device 110 via the communication interface 20. In one variant embodiment already mentioned, all processing can be at the diagnostic ECG device 110 via electronic processor 12, in which case the transmission operation 156 is omitted.

In another variant embodiment, the ECG diagnostic criteria are applied by the ECG management system 132, rather than by the diagnostic ECG device 110. In this variant embodiment, the transmit operation 156 is again omitted, as the tuned ECG diagnostic criteria are applied at the ECG management system 132.

The one or more electronic processors 12, 36 may be variously embodied, e.g. as the illustrative electronic processor 12 which together with the non-transitory storage medium 14 are assembled with the ECG device 10, 110 as a portable ECG unit, and/or as the illustrative remote electronic processor 36 and remote non-transitory storage medium 38 that are separate from the ECG device 10, 110, with the ECG device 10, 110 including a communication interface 20 via which the ECG device communicates with the remote electronic processor 36, various combinations thereof, and/or so forth. The electronic processor 12, 36 may comprise a microprocessor, graphical processing unit (GPU), and/or the like and ancillary electronics (discrete electronic components, RAM or other IC components, et cetera). The non-transitory storage medium 14, 38 may comprise a hard disk drive, RAID array or other magnetic storage medium; a solid state drive (SSD), flash memory or other electronic storage medium; an optical disk or other optical storage medium; various combinations thereof, and/or so forth.

The invention has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A cardiac device comprising:
an electrocardiograph;
an electronic processor; and
a non-transitory storage medium storing instructions readable and executable by the electronic processor to perform a process including:
receiving a location of the ECG device within a medical facility;
tuning one or more ECG analysis criteria for the received location within the medical facility to generate one or more ECG analysis criteria tuned for the received location;
operating the ECG device to acquire one or more ECG traces; and
performing cardiac analysis by applying the one or more ECG analysis criteria tuned for the received location to the one or more ECG traces to generate cardiac analysis information.

2. The cardiac device of claim 1 wherein the tuning comprises:
associating the ECG device with a medical department based on the received location; and
tuning the one or more ECG analysis criteria for the medical department with which the ECG device is associated to generate one or more ECG analysis criteria tuned for the received location.

3. The cardiac device of claim 2 wherein the tuning comprises:
referencing a first lookup table to identify a department profile corresponding to the medical department associated with the ECG device; and
referencing a second lookup table to identify ECG analysis criteria associated with the identified department profile.

4. The cardiac device of claim 1 further comprising:
an RFID tag disposed on or with the ECG device;
wherein the location of the ECG device is received from a real time locating service (RTLS).

5. The cardiac device of claim 1 wherein the non-transitory storage medium further stores a tuning look-up table associating location-specific ECG analysis criteria with specific locations, and the tuning comprises:
retrieving the one or more location-specific ECG analysis criteria for the received location from the tuning look-up table.

6. The cardiac device of claim 1 wherein the tuning comprises:
setting at least one abnormal cardiac condition detection threshold of the one or more ECG analysis criteria for the received location.

7. The cardiac device of claim 1 wherein the ECG device comprises a monitoring ECG device and the receiving comprises:
receiving identification of an assigned nurses' station to which the monitoring ECG device is assigned, wherein the location is identified as or based on the assigned nurses' station.

8. The cardiac device of claim 1 wherein the ECG device comprises a diagnostic ECG device and the tuning is further based on an ECG order.

9. The cardiac device of claim 1 wherein the ECG device comprises a diagnostic ECG device and the one or more ECG analysis criteria include at least STEMI diagnostic criteria and atrial fibrillation diagnostic criteria.

10. The cardiac device of claim 1 wherein the process further includes:
tuning the one or more ECG analysis criteria for at least one of age, gender, height, weight, body mass index, and chronic conditions such as high blood pressure, diabetes, renal failure of the monitored patient whereby the one or more ECG analysis criteria tuned for the received location are also tuned for at least one of the age and gender of the monitored patient.

11. The cardiac device of claim 1 wherein the non-transitory storage medium and the electronic processor include a non-transitory storage medium and an electronic processor assembled with the ECG device as a portable ECG unit.

12. The cardiac device of claim 1 wherein the non-transitory storage medium and the electronic processor include a remote non-transitory storage medium and a remote electronic processor that are separate from the ECG device and the ECG device includes a communication interface via which the ECG device communicates with the remote electronic processor.

13. The cardiac device of claim 1 wherein the process further includes:
outputting at least one of a cardiac analysis display and a cardiac analysis audio alarm in accord with the generated cardiac analysis information.

14. A cardiac method comprising:
receiving a location of an electrocardiograph (ECG device);
associating the ECG device with a medical department based on the received location;
tuning one or more ECG analysis criteria of the ECG device for the medical department with which the ECG device is associated to generate one or more ECG analysis criteria tuned for the associated medical department;
operating the ECG device to acquire one or more ECG traces;
performing cardiac analysis by applying the one or more ECG analysis criteria tuned for the associated medical department to the one or more ECG traces to generate cardiac analysis information;
wherein the receiving, associating, tuning, operating, and performing of cardiac analysis are performed by one or more electronic processors.

15. (canceled)

16. The cardiac method of claim 14 wherein the ECG device comprises a monitoring ECG device and the receiving of the location of the ECG device comprises: receiving identification of a nurses' station to which the ECG device is assigned, wherein the received location is identified as the assigned nurses' station.

17.-21. (canceled)
